(19) United States
(12) Patent Application Publication
(10) Pub. No.: US 2024/0299341 A1
(43) Pub. Date: Sep. 12, 2024

(54) Methods of treating or reducing symptoms of nicotine dependency (addiction) such as tobacco-use (smoking, vaping, sublingual, oral) and/or tobacco smoking cessation via combination therapy with allosteric modulators and phenethylamines.

(71) Applicant: David Alan Heldreth, JR., Bellevue, WA (US)

(72) Inventor: David Alan Heldreth, JR., Bellevue, WA (US)

(21) Appl. No.: 18/616,121

(22) Filed: Mar. 25, 2024

Related U.S. Application Data
(62) Division of application No. 17/667,147, filed on Feb. 8, 2022.
(60) Provisional application No. 63/207,183, filed on Feb. 10, 2021.

(51) Int. Cl. A61K 31/352; A61K 31/015; A61K 31/05; A61K 31/135; A61K 31/164; A61K 31/165; A61K 31/404; A61K 31/4045; A61K 31/473; A61K 31/4741; A61K 31/48; A61K 31/675; A61K 31/704; A61K 33/06; A61K 33/30; A61K 36/68; A61K 36/77

(52) U.S. Cl. CPC A61K 31/352; A61K 31/015; A61K 31/05; A61K 31/135; A61K 31/164; A61K 31/165; A61K 31/404; A61K 31/4045; A61K 31/473; A61K 31/4741; A61K 31/48; A61K 31/675; A61K 31/704; A61K 33/06; A61K 33/30; A61K 36/68; A61K 36/77

(57) ABSTRACT

The invention involves the use of formulations of positive, negative or other allosteric modulators of primarily 5ht2a receptors, but also those of: 5ht1a/b/c/d, 5ht2b/c, 5ht3, 5ht4, 5ht7, and other receptors and systems, in combination with phenethylamines and other compounds; to treat nicotine dependency (addiction) such as tobacco-use (smoking, vaping, sublingual, oral) and/or tobacco smoking cessation.

This patent (application) is a divisional patent filing which claims prior filing date/priority to patent, please reference patent title: Methods of use and formulations of allosteric modulators of the serotonin, dopamine and other receptor systems for medical, recreational, religious, research and other uses. Application Ser. No. 17/667,147—which claims prior filing date to a provisional patent. Please reference the provisional patent title: Psychedelic formulations for medical, recreational, religious, research and other uses. Application Number: 63/207,183.

### DESCRIPTION

### Field of the Invention

The invention involves the use of formulations of positive, negative or other allosteric modulators of primarily 5ht2a receptors, but also those of: 5ht1a/b/c/d, 5ht2b/c, 5ht3, 5ht4, 5ht7, in combination with phenethylamines and other compounds; to treat nicotine dependency (addiction) such as tobacco-use (smoking, vaping, sublingual, oral) and/or tobacco smoking cessation.

### Background of the Invention

People across the world are affected by nicotine dependency (addiction) such as tobacco-use (smoking, vaping, sublingual, oral) and/or tobacco smoking cessation and the serotonin system is believed to be involved in many if not all of these cases. Phenethylamines such as Mescaline, which is the active compound of many cacti, which crosses the blood brain barrier and stimulates 5-ht receptors. Phenethylamines compounds provide fast-acting and long-lasting changes to a person's illness.

However, there is variance in the effects these compounds have based on human genetics and related conditions. Allosteric binding occurs at a secondary binding site and not at the orthosteric or main agonist site and thus provides a way for additional compounds to be used in combination to provide ideal binding of receptors for individualized medicine and proper dosing. Allosteric binding affects the affinity and binding rate or activity of the receptor in a variety of ways including, but not limited to changing the shape of the receptor.

### SUMMARY OF THE INVENTION

The invention involves the use of formulations of positive, negative or other allosteric modulators of primarily 5ht2a receptors, but also those of: 5ht1a/b/c/d, 5ht2b/c, 5ht3, 5ht4, 5ht7, and other receptors and systems, in combination with phenethylamines and other compounds; to treat nicotine dependency (addiction) such as tobacco-use (smoking, vaping, sublingual, oral) and/or tobacco smoking cessation.

### BRIEF DESCRIPTION OF THE DRAWINGS

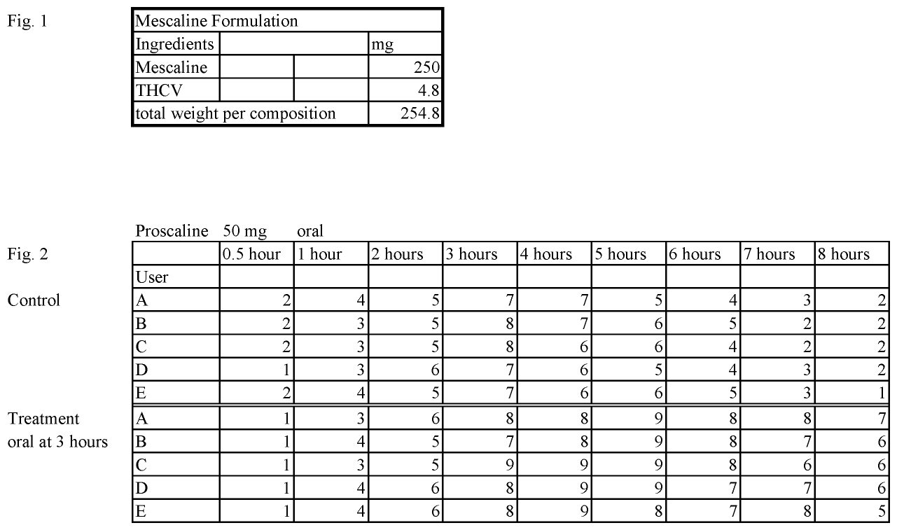

PAGE 1 drawings show potential formulations of the psychedelic compositions and the results of a human experiment on the use of proscaline alone or with positive 5HT2A allosteric modulators. PAGE 1 SHOWS FIG. 1 (FIG. 1) and FIG. 2 (FIG. 2). FIG. 1 shows a recipe for a mescaline and THCV composition to be taken orally, but may be used in other ways. FIG. 2. (FIG. 2) shows the results of a human experiment on the use of proscaline alone or with positive 5HT2A allosteric modulators.

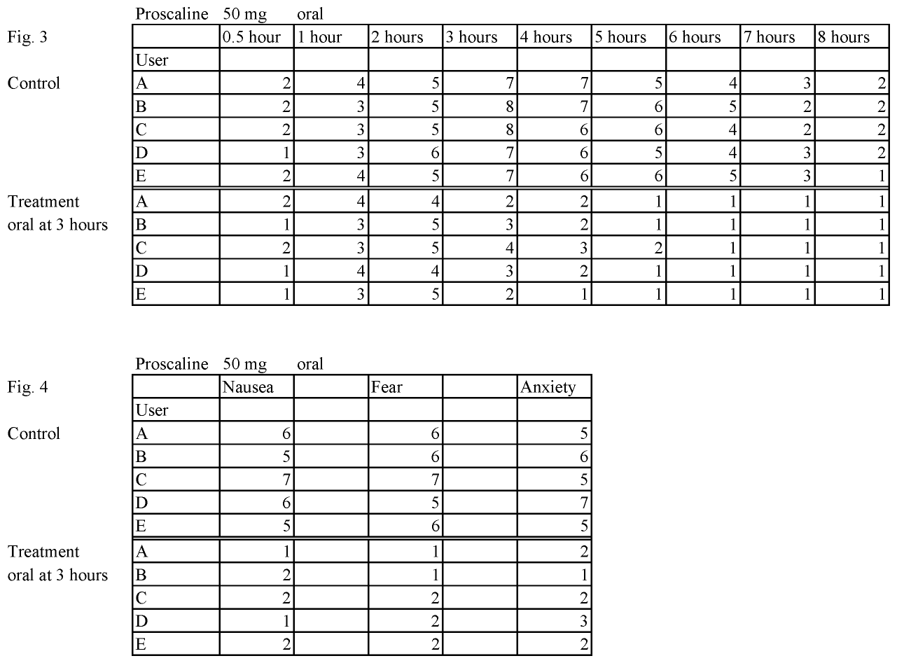

Page 2 drawings show the results of a human experiment on the use of proscaline alone or with allosteric modulators. FIG. 3. shows the results of a human experiment on the use of proscaline alone or with negative 5HT2A allosteric modulators. FIG. 4. (FIG. 4) shows the results of a human experiment on the use of proscaline alone or with allosteric modulators to modulate fear, anxiety and nausea. The compounds used in addition to the psychedelics were: for Nausea: 5ht3 allosteric modulators: linalool, citral and ginger for nausea; for anxiety and fear: 5ht7 modulator zinc and 5ht1 modulator cannabidiol.

### DRAWING SUMMARY

PAGE 1 drawings show potential formulations of the psychedelic compositions and the results of a human experiment on the use of proscaline alone or with positive 5HT2A allosteric modulators. PAGE 1 SHOWS FIG. 1 (FIG. 1) and FIG. 2 (FIG. 2). FIG. 1 shows a recipe for a mescaline and THCV composition to be taken orally, but may be used in other ways. FIG. 2. (FIG. 2) shows the results of a human experiment on the use of proscaline alone or with positive 5HT2A allosteric modulators.

Page 2 drawings show the results of a human experiment on the use of proscaline alone or with allosteric modulators. FIG. 3. shows the results of a human experiment on the use of proscaline alone or with negative 5HT2A allosteric modulators. FIG. 4. (FIG. 4) shows the results of a human experiment on the use of proscaline alone or with allosteric modulators to modulate fear, anxiety and nausea. The compounds used in addition to the psychedelics were: for Nausea: 5ht3 allosteric modulators: linalool, citral and ginger for nausea; for anxiety and fear: 5ht7 modulator zinc and 5ht1 modulator cannabidiol.

### DETAILED DESCRIPTION OF THE INVENTION

The invention involves the use of formulations of positive, negative inverse agonist, or neutral allosteric modulators of primarily 5ht2 receptors, but also those of: 5ht1a/b/c/d, 5ht2a/b/c, 5ht3, 5ht4, 5ht5, 5ht6, 5ht7, dopamine receptors (D1, D2, D3, D4, D5), adrenergic receptors (α1A, α1B, α2A, α2B, α2C, β1, β2), serotonin transporter (SERT) in combination with phenethylamines, antifungals and other items such as, but not limited to: cannabinoids, terpenes, flavonoids, minerals, compounds such as, but not limited to 5ht2a receptor agonists or other compounds; to treat nicotine dependency (addiction) such as tobacco-use (smoking, vaping, sublingual, oral) and/or tobacco smoking cessation.

Allosteric modulation is the manipulation of a receptor at a site other than normal binding site known as the orthosteric site. This is a less utilized method due to only recent discovery of its mechanisms as well as the need to identify these for each receptor. The use of allosteric modulators allows for precise alterations to the activity at the receptor and thus fine tuning of medical effects. Some embodiments utilize compounds which work as positive or negative allosteric modulators of the 5ht2a receptor or other 5ht systems. Allosteric modulation of 5ht2a also includes allosteric modulation through interaction with other receptor systems such as with heteromers or other such items.

In some embodiments the composition will include purified compounds which are either isolated or just purified. In other embodiments raw extracts or ground/processed biomass may be used. Some embodiments include excipients such as water, cyclodextrin, ethanol or other items off of the Food and Drug Administration authorized and approved excipient list.

Some embodiments will include utilizing nano technology, encapsulation, beta glucan particles, chitosan, yeast extract, surfactants, binders and other compounds to increase efficiency, availability, release lifespan, release speed among other parameters.

In some embodiments compositions eye drops, nasal spray, mouth spray, inhalers or other uses.

Some embodiments are used with vaccines, antibodies, cytokines, proteins, amino acids, DNA, RNA.

Potential compounds can be found below.

### Compound List

Phenethylamines including, but not limited to: mescaline, normescaline, 2c-i, 2c-b, 2c-e. Includes all: phenethylamines in 2(X) series such as 2-CI including all NBOME, NBOH, and other analogs.

Beta carbolines and maoi inhibitors including, but not limited to: harmaline, tetraharmaline, norharmane, perlolyrine, tetrahydroharmine, harmane, harmine, harmol.

| | | |
|---|---|---|
| 1 | AEM | alpha-Ethyl-3,4,5-trimethoxy-PEA |
| 2 | AL | 4-Allyloxy-3,5-dimethoxy-PEA |
| 3 | ALEPH | 4-Methylthio-2,5-dimethoxy-A |
| 4 | ALEPH-2 | 4-Ethylthio-2,5-dimethoxy-A |
| 5 | ALEPH-4 | 4-Isopropylthio-2,5-dimethoxy-A |
| 6 | ALEPH-6 | 4-Phenylthio-2,5-dimethoxy-A |
| 7 | ALEPH-7 | 4-Propylthio-2,5-dimethoxy-A |
| 8 | ARIADNE | 2,5-Dimethoxy-alpha-ethyl-4-methyl-PEA |
| 9 | ASB | 3,4-Diethoxy-5-methoxy-PEA |
| 10 | B | 4-Butoxy-3,5-dimethoxy-PEA |
| 11 | BEATRICE | 2,5-Dimethoxy-4,N-dimethyl-A |
| 12 | BIS-TOM | 2,5-Bismethylthio-4-methyl-A |
| 13 | BOB | 4-Bromo-2,5, beta-trimethoxy-PEA |
| 14 | BOD | 2,5,beta-Trimethoxy-4-methyl-PEA |
| 15 | BOH | beta-Methoxy-3,4-methylenedioxy-PEA |
| 16 | BOHD | 2,5-Dimethoxy-beta-hydroxy-4-methyl-PEA |
| 17 | BOM | 3,4,5, beta-Tetram ethoxy-P E A |
| 18 | 4-Br-3,5-DMA | 4-Bromo-3,5-dimethoxy-A |
| 19 | 2-Br-4,5-MDA | 2-Bromo-4,5-methylenedioxy-A |
| 20 | 2C-B | 4-Bromo-2,5-dimethoxy-PEA |
| 21 | 3C-BZ | 4-Benzyloxy-3,5-dimethoxy-A |
| 22 | 2C-C | 4-Chloro-2,5-dimethoxy-PEA |
| 23 | 2C-D | 4-Methyl-2,5-dimethoxy-PEA |
| 24 | 2C-E | 4-Ethyl-2,5-dimethoxy-PEA |
| 25 | 3C-E | 4-Ethoxy-3,5-dimethoxy-A |
| 26 | 2C-F | 4-Fluoro-2,5-dimethoxy-PEA |
| 27 | 2C-G | 3,4-Dimethyl-2,5-dimethoxy-PEA |
| 28 | 2C-G-3 | 3,4-Trimethylene-2,5-dimethoxy-PEA |
| 29 | 2C-G-4 | 3,4-Tetramethylene-2,5-dimethoxy-PEA |
| 30 | 2C-G-5 | 3,4-Norbornyl-2,5-dimethoxy-PEA |
| 31 | 2C-G-N | 1,4-Dimethoxynaphthyl-2-ethylamine |
| 32 | 2C-H | 2,5-Dimethoxy-PEA |
| 33 | 2C-I | 4-lodo-2,5-dimethoxy-PEA |
| 34 | 2C-N | 4-Nitro-2,5-dimethoxy-PEA |
| 35 | 2C-O-4 | 4-Isopropoxy-2,5-dimethoxy-PEA |
| 36 | 2C-P | 4-Propyl-2,5-dimethoxy-PEA |
| 37 | CPM | 4-Cyclopropylmethoxy-3,5-dimethoxy-PEA |
| 38 | 2C-SE | 4-Methylseleno-2,5-dimethoxy-PEA |
| 39 | 2C-T | 4-Methylthio-2,5-dimethoxy-PEA |
| 40 | 2C-T-2 | 4-Ethylthio-2,5-dimethoxy-PEA |
| 41 | 2C-T-4 | 4-Isopropylthio-2,5-dimethoxy-PEA |
| 42 | psi-2C-T-4 | 4-Isopropylthio-2,6-dimethoxy-PEA |
| 43 | 2C-T-7 | 4-Propylthio-2,5-dimethoxy-PEA |
| 44 | 2C-T-8 | 4-Cyclopropylmethylthio-2,5-dimethoxy-PEA |
| 45 | 2C-T-9 | 4-(t)-Butylthio-2,5-dimethoxy-PEA |
| 46 | 2C-T-13 | 4-(2-Methoxyethylthio)-2,5-dimethoxy-PEA |
| 47 | 2C-T-15 | 4-Cyclopropylthio-2,5-dimethoxy-PEA |
| 48 | 2C-T-17 | 4-(s)-Butylthio-2,5-dimethoxy-PEA |
| 49 | 2C-T-21 | 4-(2-Fluoroethylthio)-2,5-dimethoxy-PEA |
| 50 | 4-D | 4-Trideuteromethyl-3,5-dimethoxy-PEA |
| 51 | beta-D | beta,beta-Dideutero-3,4,5-trimethoxy-PEA |
| 52 | DESOXY | 4-Methyl-3,5-Dimethoxy-PEA |
| 53 | 2,4-DMA | 2,4-Dimethoxy-A |
| 54 | 2,5-DMA | 2,5-Dimethoxy-A |
| 55 | 3,4-DMA | 3,4-Dimethoxy-A |
| 56 | DMCPA | 2-(2,5-Dimethoxy-4-methylphenyl)- |
| | | cyclopropylamine |
| 57 | DME | 3,4-Dimethoxy-beta-hydroxy-PEA |
| 58 | DMMDA | 2,5-Dimethoxy-3,4-methylenedioxy-A |
| 59 | DMMDA-2 | 2,3-Dimethoxy-4,5-methylenedioxy-A |
| 60 | DMPEA | 3,4-Dimethoxy-PEA |
| 61 | DOAM | 4-Amyl-2,5-dimethoxy-A |
| 62 | DOB | 4-Bromo-2,5-dimethoxy-A |
| 63 | DOBU | 4-Butyl-2,5-dimethoxy-A |
| 64 | DOC | 4-Chloro-2,5-dimethoxy-A |
| 65 | DOEF | 4-(2-Fluoroethyl)-2,5-dimethoxy-A |
| 66 | DOET | 4-Ethyl-2,5-dimethoxy-A |
| 67 | DOI | 4-lodo-2,5-dimethoxy-A |
| 68 | DOM (STP) | 4-Methyl -2,5-dimethoxy-A |
| 69 | psi-DOM | 4-Methyl -2,6-dimethoxy-A |
| 70 | DON | 4-Nitro-2,5-dimethoxy-A |
| 71 | DOPR | 4-Propyl-2,5-dimethoxy-A |
| 72 | E | 4-Ethoxy-3,5-dimethoxy-PEA |
| 73 | EEE | 2,4,5-Triethoxy-A |
| 74 | EEM | 2,4-Diethoxy-5-methoxy-A |
| 75 | EME | 2,5-Diethoxy-4-methoxy-A |
| 76 | EMM | 2-Ethoxy-4,5-dimethoxy-A |
| 77 | ETHYL-J | N, alpha-diethyl-3,4-methylenedioxy-PEA |
| 78 | ETHYL-K | N-Ethyl-alpha-propyl-3,4-methylenedioxy-PEA |
| 79 | F-2 | Benzofuran-2-methyl-5-methoxy- |
| | | 6-(2-aminopropane ) |
| 80 | F-22 | Benzofuran-2,2-dimethyl-5- |
| | | methoxy-6-(2-aminoprop ane) |
| 81 | FLEA | N-Hydroxy-N-methyl-3,4-methylenedioxy-A |
| 82 | G-3 | 3,4-Trimethylene-2,5-dimethoxy-A |
| 83 | G-4 | 3,4-Tetramethylene-2,5-dimethoxy-A |
| 84 | G-5 | 3,4-Norbornyl-2,5-dimethoxy-A |
| 85 | GANESHA | 3,4-Dimethyl-2,5-dimethoxy-A |
| 86 | G-N | 1,4-Dimethoxynaphthyl-2-isopropylamine |
| 87 | HOT-2 | 2,5-Dimethoxy-N-hydroxy-4-ethylthio-PEA |
| 88 | HOT-7 | 2,5-Dimethoxy-N-hydroxy-4-(n)-propylthio-PEA |
| 89 | HOT-17 | 2,5-Dimethoxy-N-hydroxy-4-(s)-butylthio-PEA |
| 90 | IDNNA | 2,5-Dimethoxy-N,N-dimethyl-4-iodo-A |
| 91 | IM | 2,3,4-Trimethoxy-PEA |
| 92 | IP | 3,5-Dimethoxy-4-isopropoxy-PEA |
| 93 | IRIS | 5-Ethoxy-2-methoxy-4-methyl-A |
| 94 | J | alpha-Ethyl-3,4-methylenedioxy-PEA |
| 95 | LOPHOPHINE | 3-Methoxy-4,5-methylenedioxy-PEA |
| 96 | M | 3,4,5-Trimethoxy-PEA |
| 97 | 4-MA | 4-Methoxy-A |
| 98 | MADAM-6 | 2,N-Dimethyl-4,5-methylenedioxy-A |
| 99 | MAL | 3,5-Dimethoxy-4-methallyloxy-PEA |
| 100 | MDA | 3,4-Methylenedioxy-A |
| 101 | MDAL | N-Allyl-3,4-methylenedioxy-A |
| 102 | MDBU | N-Butyl-3,4-methylenedioxy-A |
| 103 | MDBZ | N-Benzyl-3,4-methylenedioxy-A |
| 104 | MDCPM | N-Cyclopropylmethyl-3,4-methylenedioxy-A |
| 105 | MDDM | N,N-Dimethyl-3,4-methylenedioxy-A |
| 106 | MDE | N-Ethyl-3,4-methylenedioxy-A |
| 107 | MDHOET | N-(2-Hydroxyethyl)-3,4-methylenedioxy-A |
| 108 | MDIP | N-Isopropyl-3,4-methylenedioxy-A |
| 109 | MDMA | N-Methyl-3,4-methylenedioxy-A |
| 110 | MDMC | N-Methyl-3,4-ethylenedioxy-A |
| 111 | MDMEO | N-Methoxy-3,4-methylenedioxy-A |
| 112 | MDMEOET | N-(2-Methoxyethyl)-3,4-methylenedioxy-A |
| 113 | MDMP | alpha,alpha, N-Trimethyl-3,4- |
| | | methylenedioxy-PEA |
| 114 | MDOH | N-Hydroxy-3,4-methylenedioxy-A |
| 115 | MDPEA | 3,4-Methylenedioxy-PEA |
| 116 | MDPH | alpha,alpha-Dimethyl-3,4-methylenedioxy-PEA |
| 117 | MDPL | N-Propargyl-3,4-methylenedioxy-A |
| 118 | MDPR | N-Propyl-3,4-methylenedioxy-A |
| 119 | ME | 3,4-Dimethoxy-5-ethoxy-PEA |
| 120 | MEDA | 3-methoxy-4,5-Ethylenedioxy-A |
| | | [Erowid corrected] |
| 121 | MEE | 2-Methoxy-4,5-diethoxy-A |
| 122 | MEM | 2,5-Dimethoxy-4-ethoxy-A |
| 123 | MEPEA | 3-Methoxy-4-ethoxy-PEA |
| 124 | META-DOB | 5-Bromo-2,4-dimethoxy-A |
| 125 | META-DOT | 5-Methylthio-2,4-dimethoxy-A |
| 126 | METHYL-DMA | N-Methyl-2,5-dimethoxy-A |
| 127 | METHYL-DOB | 4-Bromo-2,5-dimethoxy-N-methyl-A |
| 128 | METHYL-J | N-Methyl-alpha-ethyl-3,4-methylenedioxy-PEA |
| 129 | METHYL-K | N-Methyl-alpha-propyl-3,4-methylenedioxy-PEA |
| 130 | METHYL-MA | N-Methyl-4-methoxy-A |
| 131 | METHYL- | N-Methyl-2-methoxy-4,5-methylenedioxy-A |
| | MMDA-2 | |
| 132 | MMDA | 3-Methoxy-4,5-methylenedioxy-A |
| 133 | MMDA-2 | 2-Methoxy-4,5-methylenedioxy-A |
| 134 | MMDA-3a | 2-Methoxy-3,4-methylenedioxy-A |
| 135 | MMDA-3b | 4-Methoxy-2,3-methylenedioxy-A |
| 136 | MME | 2,4-Dimethoxy-5-ethoxy-A |
| 137 | MP | 3,4-Dimethoxy-5-propoxy-PEA |
| 138 | MPM | 2,5-Dimethoxy-4-propoxy-A |
| 139 | ORTHO-DOT | 2-Methylthio-4,5-dimethoxy-A |
| 140 | P | 3,5-Dimethoxy-4-propoxy-PEA |
| 141 | PE | 3,5-Dimethoxy-4-phenethyloxy-PEA |
| 142 | PEA | PEA |
| 143 | PROPYNYL | 4-Propynyloxy-3,5-dimethoxy-PEA |
| 144 | SB | 3,5-Diethoxy-4-methoxy-PEA |
| 145 | TA | 2,3,4,5-Tetramethoxy-A |
| 146 | 3-TASB | 4-Ethoxy-3-ethylthio-5-methoxy-PEA |
| 172 | 5-TOM | 2-Methoxy-4-methyl-5-methylthio-A |
| 173 | TOMSO | 2-Methoxy-4-methyl-5-methylsulfinyl-A |
| 174 | TP | 4-Propylthio-3,5-dimethoxy-PEA |
| 175 | TRIS | 3,4,5-Triethoxy-PEA |
| 176 | 3-TSB | 3-Ethoxy-5-ethylthio-4-methoxy-PEA |
| 177 | 4-TSB | 3,5-Diethoxy-4-methylthio-PEA |
| 178 | 3-T-TRIS | 4,5-Diethoxy-3-ethylthio-PEA |
| 179 | 4-T-TRIS | 3,5-Diethoxy-4-ethylthio-PEA |

Additionally includes all: (4-acetoxy) (4-hydroxy)(dimethyl) (diethyl) (N-methyl-N-ethyl) (N-methyl)(N-methyl-N-isopropyl)(N,N-diisopropyl) variations of compounds in compound list.

Additionally includes all: functional group variants, fumerates, fumerics, idoines, hydrofumarates, deneutered or not, salts, acids, isomers, analogs, precursors, further metabolites of biosynthetic or synthetic pathways.

Also: Zinc, Magnesium, Sulfate, Carvelidol.

1. The method of treating or reducing symptoms of nicotine dependency (addiction) such as tobacco-use (smoking, vaping, sublingual, oral) and/or tobacco smoking cessation, consisting of:
Administering to a human patient a therapeutic amount of a composition comprising:
a 5ht receptor allosteric modulator or modulators, selected from: THCV (Tetrahydrocannabivarin), oleamide, linalool, limonene, tetrahydrocannabinol, cannabidiol or zinc; AND
One item or a mixture of: mescaline, hordenine, anhalonidine, pellotine or other phenethylamine; AND OPTIONALLY
Administering to a human patient a therapeutic amount of a composition comprising: A 5ht, allosteric modulator or modulators selected from: palmitoleamide, 2,2-dimethyloleamide, N-oleoyl glycine, myristoleamide, 1-oleyl-2-acetylglycerol, anandamide, oleyl aldehyde, trifluoromethyl ketone, oleic acid, oleylpropanolamide alpha-pinene, cannabidiol, carvelidol.

2. The method of claim 1 including further administering to a human patient a therapeutic amount of a composition comprising: a vaccine or antibodies which target nicotine or its metabolites.

3. The method of claim 1 including testing/diagnosing the human patient such as, but not limited to: genetic testing.

4. The method of claim 1 including administering to a human patient a therapeutic amount of a composition comprising either: harmine, hamaline, or other harmala alkaloids or a combination thereof.

5. The method of claim 1 including administering to a human patient a therapeutic amount of a composition comprising either: valencene, borneol, alpha-pinene, limonene, linalool, salivorin-A, other terpenes/terpenoids or a mixture thereof.

6. The method of claim 1 including administering to a human patient a therapeutic amount of an iboga alkaloids such as, but not limited to:
Coronaridine, lbogamine, Voacangine, Noribogaine or lysergides such as, but not limited to LSD, LSZ, 1-PLSD, ALD-52, 1V-LSD, or other analogs.

7. The method of claim 1 including administering to a human patient a therapeutic amount of one item or a mixture of: 4-ho-Met, 5-MEO-Met, DipT, 4-ho-DiPT, 5-MEO-AMT, 5-MEO-MiPT, 5-MEO-DET, 4-ho-DET, or other tryptamine.

8. The method of claim 1 including a step of administering to a human patient a therapeutic amount of: an allosteric modulator of the 5ht3, 5ht4, or 5ht7 receptors.

9. The method of claim 1 including a step of administering to a human patient a therapeutic amount of an allosteric modulator of GLP-1.

10. The method of claim 1 including a step of administering to a human patient a therapeutic amount of: an allosteric modulator of a dopamine receptor.

11. The method of treating or reducing symptoms of nicotine dependency (addiction) such as tobacco-use (smoking, vaping, sublingual, oral) and/or tobacco smoking cessation, consisting of:
Administering to a human patient a therapeutic amount of a composition comprising: a vaccine or antibodies which target nicotine or its metabolites.
Administering to a human patient a therapeutic amount of a composition comprising:
a 5ht receptor allosteric modulator or modulators, selected from: THCV (Tetrahydrocannabivarin), oleamide, linalool, limonene,
tetrahydrocannabinol, cannabidiol or zinc; AND One item or a mixture of: proscaline (4-propoxy-3,5-dimethoxyphenethylamine or 4-propoxy-3,5-DMPEA), N-methylmescaline, N,N-Diformylmescaline, methylmescaline, normescaline, n-acetylmescaline, 2C-B, 2C-C, 2C-D, 2C-E, 2C-I, 2C-P, 2C-T, 2C-T2, 2C-T4, 2C-T7, 2C-T9, Trimethoxyamphetamine, or 3,4-Methylenedioxy methamphetamine; AND OPTIONALLY
Administering to a human patient a therapeutic amount of a composition comprising: A 5ht, allosteric modulator or modulators selected from: palmitoleamide, 2,2-dimethyloleamide, N-oleoyl glycine, myristoleamide, 1-oleyl-2-acetylglycerol, anandamide, oleyl aldehyde, trifluoromethyl ketone, oleic acid, oleylpropanolamide alpha-pinene, cannabidiol, carvelidol.

12. The method of claim 11 including testing/diagnosing the human patient such as, but not limited to: genetic testing.

13. The method of claim 11 including administering to a human patient a therapeutic amount of a composition comprising either: harmine, hamaline, or other harmala alkaloids or a combination thereof.

14. The method of claim 11 including administering to a human patient a therapeutic amount of a composition comprising either: valencene, borneol, alpha-pinene, linalool, limonene or a mixture thereof.

15. The method of claim 11 including administering to a human patient a therapeutic amount of a vaccine or antibodies which target nicotine or its metabolites.

16. The method of claim 11 including a step of administering to a human patient a therapeutic amount of: an allosteric modulator of the 5ht3, 5ht4, or 5ht7 receptors.

17. The method of claim 11 including a step of administering to a human patient a therapeutic amount of an allosteric modulator of GLP-1.

18. The method of claim 11 including administering to a human patient a therapeutic amount of an iboga alkaloids such as, but not limited to: Coronaridine, lbogamine, Voacangine, Noribogaine or lysergides such as, but not limited to LSD, LSZ, 1-PLSD, ALD-52, 1V-LSD, or other analogs.

19. The method of claim 11 including administering to a human patient a therapeutic amount of one item or a mixture of: 4-ho-Met, 5-MEO-Met, DipT, 4-ho-DiPT, 5-MEO-AMT, 5-MEO-MiPT, 5-MEO-DET, 4-ho-DET, or other tryptamine.

20. The method of claim 11 including a step of administering to a human patient a therapeutic amount of: an allosteric modulator of a dopamine receptor.
